(19) United States
(12) Patent Application Publication
(10) Pub. No.: US 2023/0176034 A1
(43) Pub. Date: Jun. 8, 2023

(54) Engineered PlyAB Nanopores and Uses Thereof

(71) Applicant: Rijksuniversiteit Groningen, Groningen (NL)

(72) Inventors: Giovanni Maglia, Glimmen (NL); Gang Huang, Groningen (NL); Mikhael Soskine, Morigny-Champigny (FR)

(21) Appl. No.: 17/905,911

(22) PCT Filed: Mar. 11, 2021
(86) PCT No.: PCT/NL2021/050167

§ 371 Date: Sep. 8, 2022

(30) Foreign Application Priority Data
Mar. 11, 2020 (EP) 20162431.9

(51) Int. Cl. G01N 33/487

(52) U.S. Cl. CPC G01N 33/48721; B82Y 40/00

(57) ABSTRACT

The invention relates generally to the field of nanopores and the use thereof in analyzing biopolymers. In particular, it relates to engineered biological nanopores and their application in single molecule analysis, such as single molecule protein identification.

The invention relates generally to the field of nanopores and the use thereof in analyzing biopolymers. In particular, it relates to engineered biological nanopores and their application in single molecule analysis, such as single molecule protein identification.

Nanopores hold great potential for studying biomolecules and much initial effort focused on the detection of unfolded polymers such as PEG [1, 2, 3], DNA [4, 5, 6, 7, 8], unfolded proteins [9, 10] or peptides [11, 12, 13, 14, 15, 16, 17, 18, 19, 20], or small analytes [20]. Biological nanopore sensors typically consist of a nanometer-sized, protein-based pore embedded in an insulating membrane that separates two chambers filled with an electrolyte solution. When an electrical bias is applied across the membrane, ions will flow through the pore, producing an open pore current. Molecules traversing the pore under such an external potential will temporarily block or reduce the flow of ions, with this effect being more pronounced when the traversing molecule is relatively large compared to the pore diameter. This change in ionic current can be measured, allowing single molecule identification and characterization of unlabeled analytes, in real-time and under physiological conditions. Notably, biological nanopores are now routinely used to sequence nucleic acids at the single molecule level.

Despite finding wide-spread use in the analysis of nucleotides and small or unfolded peptides, the applicability of biological nanopores to study folded proteins has so far been limited. The main reason for this is that the diameter of most known biological nanopores is too small for most folded proteins to enter into and/or translocate through the pore.

Recently, the applicant has developed two a-helical biological nanopores, fragaceatoxin C (FraC) (EP 3485029 A1) and cytolysin A (ClyA) (EP 2978773 A1), which are suitable for detection of peptides and small proteins. In particular, wild type or engineered ClyA pores, consisting of 12 ClyA monomers resulting in a constriction diameter of 3.3 nm, can detect folded proteins with a molecular weight up to approximately 40 kDa. Alternative ClyA pores may comprise 13 or 14 monomers. The largest of the resulting pores has a constriction diameter of 4.2 nm. However, this pore is not efficient or stable enough for electrophysiological sensing applications.

As an alternative to biological nanopores, solid state nanopores may be used to study folded proteins. Despite being in principle capable of sensing proteins ranging in size from approximately 6 to 660 kDa, such artificial nanopores suffer from many drawbacks. Proteins, with their non-uniform charge distribution, often adsorb to the nanopore surface or translocate too quickly to be sampled properly. It also remains challenging to reproducibly manufacture solid-state nanopores of uniform size, which is essential for reliable detection. Moreover, compared to a biological nanopore, it is not straightforward to modify the surface properties inside the pore to optimize detection. In particular, the surface charge, which controls the nanofluidic properties of the nanopore [21, 22, 23], cannot be modified with atomic precision, and binding elements cannot be introduced with controlled stoichiometry.

The inventors recognized the need for a nanopore capable of detecting larger (>~40 kDa) folded proteins, which nanopore can also be easily and reproducibly manufactured and applied for commercial electrophysiological sensing applications.

In particular, the goal of the invention is to provide a uniformly sized nanopore with a sufficiently large constriction diameter and an appropriate selectivity to allow entry of large, folded proteins. Preferably, the pore is sufficiently stable under conditions used for electrophysiological sensing experiments. Furthermore, in order to enable reliable electrophysiological sensing experiments, it preferably does not display significant spontaneous opening and closing within the relevant experimental timescales.

Therefore, the inventors set out to develop a novel type of nanopore by engineering properties such as stability and ion selectivity of a biological nanopore. To that end, they used a unique combination of directed evolution and site-specific mutagenesis to tune the properties of a biological β-barrel nanopore.

This approach surprisingly resulted in the provision of a β-barrel biological nanopore having a cylindrical trans chamber and a truncated cone cis chamber, separated by an inner constriction with a diameter of at least approximately 2 nm being capable of detecting large, folded proteins, including for example the 66.5 kDa human albumin and the 76-81 kDa human transferrin proteins. More in particular, it was found that pleurotolysin (Ply) A and B subunits can be genetically engineered to form a stable pore of such dimensions with tunable ion-selectivity.

Accordingly, the invention provides a β-barrel biological nanopore having a cylindrical trans chamber and a truncated cone cis chamber, separated by an inner constriction with a diameter of at least approximately 2 nm. Preferably, such a nanopore is an assembly of genetically engineered pleurotolysin (Ply) A and B subunits, more preferably it is an assembly of 26 PlyA subunits or monomers and 13 PlyB subunits or monomers. Furthermore, the use of such nanopores to detect biomolecules or complexes thereof, particularly large, folded proteins, is provided.

A nanopore of the invention or its use in the detection of biomolecules is not known or suggested in the art.

A large number of β-barrel biological nanopores can be found in nature. Stable nanopores suitable for sensing applications have been obtained for comparatively small β-barrel pores, with typical diameters of approximately 1-2 nm. Such small nanopores may be functionalized by introducing a recognition element at the pore entrance to enable protein detection (e.g. WO 2019/158548 A1, US 2019/0128867 A1). This approach is clearly limited by the extra modification to include a sensing element which will need optimization for each target protein. Moreover, as detection takes place outside the pore, full characterization of the target protein may be hampered and a single protein may be detected multiple times as it will not translocate through the pore.

To date, the largest β-barrel pore suitable for protein sensing is a perforin nanopore [24]. This nanopore is capable of sensing proteins with a molecular weight up to about 28 kDa. However, proteins can only pass through the pore in an unfolded state. Moreover, these nanopores have a broad size distribution, making them unattractive for commercial development.

Thus, the prior art fails to disclose or suggest a β-barrel biological nanopore of the invention. Moreover, the use of such a nanopore to detect biomolecules, in particular folded proteins or complexes thereof with a molecular weight over about 40 kDa, is not anticipated.

In one embodiment, the invention provides a nanopore which is an assembly of genetically engineered pleurotolysin (Ply) A and B subunits.

Pleurotolysin from the mushroom Pleurotus ostreatus belongs to the Membrane Attack Complex PerForin/Cholesterol Dependent Cytolysin (MACPF/CDC) protein superfamily [25]. Members of this large and diverse superfamily are found in all kingdoms of life and are involved in a wide variety of processes, including vertebrate immunity, venom toxicity, neural development and plant pathogen defense. Other example MACPF/CDC pore forming toxins include perforin, complement C9, pneumolysin and lysteriolysin.

Members of the MACPF/CDC superfamily form unusually large pores, comprising up to about 50 monomers resulting in pore diameters up to 30 nm, in comparison to other known multimeric β-barrel pores, which typically comprise 7-9 monomers. For example, perforin pores typically comprise 18-25 monomers.

The fungal MACPF/CDC pleurotolysin is a bi-component system composed of PlyA and PlyB subunits. Only in concert do PlyA and PlyB exhibit cytolytic activity characteristic of pore formation. PlyA is responsible for membrane recognition and binding, whereas the transmembrane β-barrel is formed by PlyB. PlyA specifically targets sphingomyelin lipids of cholesterol enriched membranes.

Structures of the PlyA and B monomers, PlyAB pores and pore intermediates have recently been elucidated [26] using a combination of biophysical, crystallographic and SP cryo-EM methods. The majority of PlyAB pores are formed by 26 PlyA subunits, which bind the membrane as dimers upon which 13 PlyB monomers can assemble and undergo the required conformational transition to form a transmembrane β-barrel. The resulting pore is approximately 10 nm tall and has an overall diameter of approximately 8 nm, making it the narrowest MACPF/CDC pore known to date. A 3D structure of the PlyAB pore is shown in FIG. 1, where it is also compared to two other common protein pores.

As shown in FIG. 1, the PlyAB pore comprises a cylindrical trans chamber with a diameter of ~7.2 nm attached to a truncated cone cis chamber with a larger diameter of approximately 10.5 nm. The pore is at its narrowest where the two chambers meet and the diameter at this constriction zone is approximately 5.5 nm. Blockades of the pore by an analyte, such as a folded protein, generally occur at the constriction zone.

In a preferred embodiment a nanopore of the invention is an assembly of 26 genetically engineered PlyA monomers and 13 genetically engineered PlyB monomers.

The sequence of the wild type PlyA polypeptide can be accessed from the Uniprot database using the Pfam Q8X1M9 identifier. The PlyA sequence shown in FIG. 2A is identical to the Pfam Q8X1M9 sequence, with an additional GSA-linked C-terminal His6-tag. For the discussion of mutations in genetically engineered PlyA subunits for use in the invention herein below, the numbering of the sequence as shown in FIG. 2A is adhered to. This numbering corresponds to the residue numbering in Pfam Q8X1M9. For example, residue C62 of FIG. 2A corresponds to residue C62 of Pfam Q8X1M9.

In one embodiment, the sequence into which mutations are introduced to obtain a PlyA monomer or polypeptide for use in a nanopore of the invention has at least 80%, preferably at least 85%, more preferably at least 90% sequence identity with Pfam Q8X1M9. Particularly preferred is a PlyA polypeptide with a sequence identity between 95% and 100%, for instance 96%, 97%, 98% or 99%, with Pfam Q8X1M9.

Optionally, extra residues may be included at the C-terminus and/or N-terminus of a PlyA monomer for use in the invention. For instance, an affinity tag, such as a His-tag or Strep-tag, is added to the N- or C-terminus. Preferably the affinity tag is a His-tag. More preferably, the His-tag is fused to the C-terminus, most preferably via a short, flexible linker. For example, the sequence of FIG. 2a includes a C-terminal His6-tag attached via a GSA linker. Accordingly, in a preferred embodiment, the sequence into which mutations are introduced to obtain a genetically engineered PlyA monomer for use in the invention corresponds to FIG. 2A.

The sequence of the full-length wild type PlyB polypeptide can be accessed from the Uniprot database using identifier Pfam Q5W9E8. The polypeptide sequence shown in FIG. 3A contains residues 49 to 523 of Pfam Q5W9E8, flanked by an N-terminal sequence MA and a C-terminal GSA-linked His6-tag. Residues 49-523 of Pfam Q5W9E8 largely correspond to the part of the protein for which the 3D structure has been elucidated and deposited with PDB identifier 40EJ [26]. For the discussion herein below of mutations introduced to obtain genetically engineered PlyB monomers for use in the invention, the residue numbering of Pfam Q5W9E8 is adhered to. Table 1 shows the correspondence between the amino acid residue numbers of the Pfam sequence Q5W9E8 and FIG. 3a.

TABLE 1

Comparison between residue numbering according to Pfam Q5W9E8 and the sequence of FIG. 3a for the PlyB mutations of the present invention

| | |
|---|---|
| Pfam Q5W9E8 | FIG. 3a |
| N72 | N26 |
| N153 | N107 |
| G264 | G218 |
| K301 | K255 |
| E306 | E260 |
| E307 | E261 |
| E316 | E270 |
| A374 | A328 |
| C487 | C441 |
| A510 | A464 |

In one embodiment, a PlyB monomer or polypeptide for use in a nanopore of the invention consists of residues 49 to 523 of Pfam Q5W9E8, prior to the introduction of mutations. In another embodiment, the PlyB sequence into which mutations are introduced to provide PlyB monomers for use according to the invention, has at least 80%, preferably at least 85%, more preferably at least 90% sequence identity with residues 49-523 of Pfam Q5W9E8. Most preferably, the PlyB sequence has between 95 and 100%, for instance 96%, 97%, 98% or 99%, sequence identity with the sequence corresponding to residues 49-523 of Pfam Q5W9E8. Preferably, residues 405-408, 466-470 and 497-504, which are thought to be involved in PlyB-PlyA interaction, are conserved.

Optionally, extra residues may be added to the C-terminus and/or N-terminus of a PlyB monomer for use in the invention. For instance, an affinity tag, such as a His-tag or Strep-tag, is added to the N- or C-terminus. Preferably the affinity tag is a His-tag. More preferably, the His-tag is fused to the C-terminus, most preferably via a short, flexible linker. For example, the sequence shown in FIG. 3a includes a C-terminal His6-tag attached via a GSA linker. N-terminally, the PlyB sequence can be extended such that a larger selection of the Pfam Q5W9E8 sequence is included. For instance, the PlyB sequence may comprise residues 47-523, residues 45-523, residues 40-523, residues 32-523, residues 25-523, residues 20-523, residues 10-523 or residues 1-523 of Pfam Q5W9E8. Additional N-terminal amino acids may also result from a cloning strategy used to produce the PlyB monomers. For example, in FIG. 3A the sequence MA preceding residues 49-523 of Pfam Q5W9E8 stems from the introduction of a NcoI restriction site into the DNA sequence, along with two extra bases to maintain the reading frame.

The PlyB sequence of FIG. 3a contains residues 49-523 of Pfam Q5W9E8 with an additional N-terminal MA sequence and a C-terminal GSA-linked His6-tag. In a specific aspect of the invention, the sequence into which mutations are introduced to obtain a genetically engineered PlyB monomer for use in the invention is that of FIG. 3A. In another embodiment, a PlyB monomer for use in the invention has at least 80%, preferably at least 85%, more preferably at least 90%, most preferably at least 95% sequence identity with the sequence according to FIG. 3a, prior to the introduction of mutations.

As will be understood by one skilled in the art, PlyA and/or PlyB monomers for use in a nanopore according to the invention may comprise one or more conservative mutations. Typically, conservative mutations wherein an amino acid is replaced by a residue with very similar properties are anticipated to have no or only a limited effect on nanopore function. Examples of conservative mutations include S to T, R to K, D to E, N to Q, A to V, I to L, F to Y and vice versa.

Pore opening and closing in the absence of potential analytes, also known as spontaneous gating, severely limits a nanopore’s usefulness for electrophysiological sensing.

Therefore, in one embodiment, the invention provides a nanopore comprising PlyA monomers engineered to form stable pores which remain open for prolonged periods even under an applied transmembrane potential. Examples include a pore for which no spontaneous gating is observed within a period of at least 10, 20, 30, or 60 s at an applied potential of -50 mV or for which no spontaneous gating is observed within a period of 5, 10, 15, or 20 s at an applied potential of -150 mV.

WT PlyA monomers contain two cysteines: C62 and C94. Both these cysteines are in the region of the protein which interacts with the lipid bilayer and may therefore be involved in membrane anchoring. As membrane anchoring may be linked to pore stability, it is hypothesized that pore stability may be adjusted by mutating one or both of the cysteine residues.

In a specific embodiment, a nanopore comprises PlyA monomers wherein C62 is replaced with another amino acid. Alternatively, C94 of the PlyA subunits is substituted. In a preferred embodiment, the nanopore comprises PlyA subunits wherein both C62 and C94 are substituted. Without wishing to be bound by theory, the oxidative nature of the cysteine residues may be responsible for their destabilizing effect on the nanopore. Thus, any non-oxidizing, i.e. non-negatively charged, amino acid may be substituted for one or both of the cysteines. For instance, cysteine replacements may be selected from glycine, alanine, valine, leucine, isoleucine, serine, threonine, asparagine and glutamine, lysine, arginine, phenylalanine, tyrosine or tryptophan. In a preferred embodiment, C62 and/or C94 replacements are individually selected from alanine, serine and threonine. For example, PlyA monomers for use in the present invention comprise either the mutations C62A and C94A, C62A and C94S, or C62A and C94T. As another example, PlyA monomers comprise the mutations C62T and C94A, C62T and C94S, C62T and C94T, C62S and C94A, C62S and C94S, or C62S and C94T.

As shown in Example 4, PlyAB-E1, PlyAB-E2, and PlyAB-R pores display desirable stability. These nanopores all comprise PlyA subunits wherein both cysteines are substituted by serine. Hence, in a particularly preferred embodiment, the nanopore comprises PlyA monomers with the mutations C62S and C94S. PlyA monomers comprising both C62S and C94S mutations are referred to as PlyA-S in the Examples.

An attractive way to obtain large quantities of PlyA and PlyB subunits for use in a nanopore of the invention is to express the monomers in a suitable host, for instance E. coli. Whilst PlyA variants of interest can easily be obtained from such a set up, PlyB monomers have limited solubility and typically end up in inclusion bodies. Although it is possible to refold PlyB as part of a protein purification routine, this is not attractive or even feasible for large scale production necessary for commercial nanopore exploitation.

Therefore, in one aspect, the invention provides a nanopore wherein the PlyB monomers comprise at least one mutation that increases the solubility of this subunit. Such a mutation contributing to improved water-solubility may be termed a “solubility-enhancing mutation”. Solubility enhancing mutations typically involve amino acids whose sidechains are solvent-exposed when PlyB is in its monomeric folded state. Generally, improved solubility may be obtained by replacing one or multiple of such solvent-exposed amino acids with a more hydrophilic amino acid. For example, leucine, isoleucine, valine or alanine may be replaced with serine, threonine, glutamine, asparagine, arginine, lysine, aspartic acid or glutamic acid. Or serine, threonine, glutamine or asparagine may be replaced by arginine, lysine, aspartic acid or glutamic acid.

In a particular embodiment, PlyB N72 is substituted by aspartic acid (N72D) or glutamic acid (N72E). Alternatively, PlyB A374 is replaced with serine (A374S) or threonine (A374T). In a preferred embodiment, a nanopore comprises PlyB subunits with the mutations N72D/E and A374S/T, preferably N72D and A374T.

In another embodiment, PlyB monomers furthermore comprise one or more of the mutations N153D/E and/or G264R/K. In a preferred embodiment, PlyB subunits comprise both mutations N153D/E and G264R/K, together with the above mentioned solubility enhancing mutations N72D/E and A374S/T. In a particularly preferred embodiment, PlyB subunits comprise the mutations N72D, N153D, G264R and A374T.

PlyB mutants with enhanced solubility may be obtained by introducing point-mutations through site-directed mutagenesis. A preferred approach, however, is to obtain such mutants through a directed evolution approach as described herein in the Experimental section.

In some instances, mutant PlyB monomers obtained or further developed through one or multiple rounds of directed evolution comprise further mutations which do not prima facie seem to contribute to solubility of the protein. In particular, one or more amino acids may be replaced with a more hydrophobic residue. For instance, A510 may be replaced with valine, leucine or isoleucine, preferably valine. Without wishing to be bound by theory, such substitutions are likely to contribute to stability of the folded monomeric PlyB protein and possibly compensate for a degree of destabilization arising from the introduction of more hydrophilic, or even charged, residues. Such mutations can be referred to as ancillary mutations.

Thus, in one embodiment, a nanopore comprising PlyB subunits with at least one solubility enhancing mutation and one or more ancillary mutations. For instance, in a specific aspect, a nanopore comprises PlyB subunits with the mutations N72D/E, A374S/T and A510V/I/L. The Examples highlight that a PlyAB-E1 nanopore comprising PlyB monomers with the mutations N72D, A374T and A510V was capable of distinguishing between two 64 kDa protein tetramer differing only in a point mutation. Hence, in a preferred embodiment PlyB subunits for use in the invention comprise the mutations N72D, A374T and A510V.

In another embodiment, a nanopore comprises PlyB monomers with the mutations N72D/E, N153D/E, G264R/K, A374T/S and A510V/I/L. Preferably, the PlyB monomer comprises the mutations N72D, N153D, G264R, A374T and A510V.

In some embodiments, PlyA and/or PlyB subunits may comprise other mutations. For example, C487 of PlyB may be replaced with alanine, serine or threonine, preferably with alanine. PlyB monomers used in PlyAB-E2 pores, which display the ability to capture a range of folded proteins as shown herein in the Examples, comprise the mutations N72D, N153D, G264R, A374T, A510V and C487A. Therefore, in a preferred embodiment, PlyB monomers for use in the invention comprise the mutations N72D, N153D, G264R, A374T, A510V and C487A.

The ion selectivity of a PlyAB nanopore may affect its ability to detect a wide range of large, folded proteins. WT PlyAB pores and PlyAB pores comprising the mutations discussed herein above display a high density of negatively charged amino acids around the constriction site. Hence, these pores are slightly cation-selective and appear to selectively allow the passive diffusion of small cationic proteins, such as granzyme B, over neutral and anionic proteins.

A cation-selective PlyAB-based pore comprising one or both of the cysteine mutations according to the invention is capable of detecting certain large folded proteins. However, for some applications, an anion selective pore may be more desirable.

It was surprisingly found that the electroosmotic flow can be reduced or inverted by increasing the net positive charge of the inner surface of the nanopore. For instance, an anion-selective PlyAB pore can be obtained by replacing some or all of the negatively charged amino acids E306, E307 and E316 in the constriction site by positively charged ones.

Therefore, in one aspect, a nanopore comprises PlyB monomers wherein the net positive charge of the inner surface of the nanopore has been increased. For example, one or more negatively charged residues are exchanged for neutral or positively charged residues. Alternatively, one or more neutral (preferably polar) amino acids are replaced with a positively charged amino acid. Preferably, the net positive charge of the inner surface of the constriction zone is increased. More preferably, a PlyB monomer for use in an anion-selective nanopore of the invention comprises at least one of the mutations E306K/R, E307K/R or E316K/R. For instance, E306 is replaced with either lysine or arginine. Alternatively, E307 or E316 of the PlyB monomer is substituted by lysine or arginine.

In a preferred embodiment, two of the aforementioned glutamates are replaced. In one aspect PlyB monomers comprise the mutations E306K/R and E307K/R. In another aspect, PlyB monomers comprise the mutations E306K/R and E316K/R. In yet another embodiment, PlyB subunits comprise the mutations E307K/R and E316K/R.

PlyAB-R pores wherein PlyB monomers comprise the mutations E306R, E307R and E316R have been shown to be particularly effective at detecting large, folded proteins and are capable of distinguishing different human plasma proteins. Thus, in a particularly preferred embodiment, PlyB monomers comprise the mutations E306K/R, E307K/R and E316K/R, wherein the replacements are independently selected. In an even more preferred embodiment, PlyB monomers for use in a nanopore of the invention comprise the mutations E306R, E307R and E316R.

In a further aspect, PlyB monomers comprising one or more mutations which increase the net number of positive charges in the constriction site, also comprise at least one solubility enhancing mutation. Preferably, such PlyB monomers also comprise at least one ancillary mutation. In a preferred embodiment, PlyB monomer comprising at least one of the mutations E306K/R, E307K/R and E316K/R also comprise one or more of the solubility enhancing mutations discussed herein above. In a particularly preferred embodiment, PlyB subunits comprising at least one of the mutations E306K/R, E307K/R and E316K/R also comprise the mutations N72D/E and A374T/S, optionally together with the mutation A510V/I/L. A PlyB monomer comprising the mutations E306R, E307R, E316R, N72D, A374T and A510V is even more preferred.

Even in the presence of the above mentioned solubility-enhancing mutations, PlyB monomers comprising one or more of the E to R/K substitutions display reduced solubility. Hence, such mutations are ideally combined with one or more mutations in PlyB which compensate for this effect. For instance, introduction of the mutation K301E was found to improve the solubility of PlyB mutants further comprising the mutations N72D, E306R, E307R, E316R, A374T and A510V.

Hence, in one embodiment, PlyB monomers comprise the mutation K301D/E in combination with one or more mutations selected from the group E306K/R, E307K/R and E316K/R and one or more of the mutations N72D/E and/or A374S/T. In a preferred embodiment, PlyB monomers comprising at least two of the mutations selected from the group E306K/R, E307K/R and E316K/R and one or more of the mutations N72D/E and/or A374S/T, further comprise the mutation K301D/E. Even more preferred are PlyB monomers comprising the K301D/E mutation in combination with all three of the mutations E306K/R, E307K/R and E316K/R, and the mutations N72D/E and A374S/T. In a particularly preferred embodiment, PlyB subunits comprise the mutations E306R/K, E307R/K, E316R/K, N72D/E, A374T/S, A510V/I/L and K301D/E, more preferably the mutations E306R, E307R, E316R, N72D, A374T, A510V and K301E.

As shown herein below, the cation-selective PlyAB-E1 nanopore comprising PlyA subunits with the mutations C62S and C94S along with PlyB subunits with the mutations N72D, A374T and A510V displays good pore stability, ability to detect a range of large folded proteins and ease of production due to improved soluble expression of the PlyB monomers. Hence, in one embodiment the invention provides a nanopore comprising PlyA subunits with the mutations C62S and C94S, and PlyB subunits with the mutations N72D, A374T and A510V.

The related cation-selective PlyAB-E2 nanopore, wherein PlyB subunits furthermore comprise the mutations N153D, G264R and C487A, was also found to be capable of detecting folded proteins. Hence in another embodiment, the invention provides a nanopore comprising PlyA subunits with the mutations C62S and C94S, and PlyB subunits with the mutations N72D, N153D, G264R, A374T, C487A and A510V.

An anion-selective nanopore which shows a particularly advantageous combination of good pore stability, anion selectivity and ease of production comprises PlyA subunits with the mutations C62S and C94S along with PlyB subunits with the mutations N72D, K301E, E306R, E307R, E316R, A374T, C487A and A510V. This pore is referred to as PlyAB-R in the Examples. Therefore, in a further aspect, a nanopore comprising PlyA subunits with the mutations C62S and C94S along with PlyB subunits with the mutations N72D, K301E, E306R, E307R, E316R, A374T, C487A and A510V is provided.

Also provided are PlyA and PlyB polypeptides comprising one or more of the mutations described above. In a preferred embodiment, a His-tag of six or more residues is attached to the polypeptide for ease of purification. Such a His-tag is optimally attached to the C-terminus of the polypeptide via a GSA linker. Such mutant PlyA and PlyB polypeptides are advantageously used in a nanopore according to the invention.

The invention furthermore provides an isolated nucleic acid molecule encoding a mutant PlyA or PlyB monomer according to the invention as well as an expression vector comprising the nucleic acid molecule. Still further, the invention provides a host cell comprising said expression vector.

In a specific aspect, a system comprising a nanopore according to the invention assembled into a lipid bilayer is provided. Preferable, multiple such systems are integrated into a device. Such a device is ideally portable and can contain hundreds or thousands of individual sensors, enabling high-throughput single molecule detection. Accordingly, in one embodiment the invention provides A portable device comprising a plurality of individual systems, each system comprising a nanopore according to the invention assembled into a lipid bilayer. For example, individual nanopores according to the invention embedded in a membrane are set in an arrayed sensor chip, wherein each sensing unit corresponds to its own electrode that is connected to a channel in the sensor array chip. In this setup, each nanopore channel is controlled and measured individually by a bespoke application-specific integrated circuit (ASIC), allowing for multiple nanopore experiments to be performed in parallel. Such devices, for instance Oxford Nanopore’s MinION and PromethION, are now widely used for DNA and RNA sequencing. The present invention provides similar devices for protein analysis.

A further embodiment relates to a method for providing such a system comprising a nanopore according to the invention assembled into a lipid bilayer, comprising the steps of
providing mutant PlyA polypeptides;
providing mutant PlyB polypeptides;
contacting said mutant PlyA polypeptides with liposomes under conditions allowing for association of PlyA and liposome to form PlyA-liposomes; followed by
contacting said PlyA-liposomes with said mutant PlyB polypeptides resulting in the formation of PlyAB lipoprotein complex; and subsequently
contacting the lipoprotein complex with a lipid bilayer to allow formation of nanopores.

Alternatively, a method of preparation involves surfactants instead of liposomes. In this case, mutant PlyA monomers are contacted with surfactant micelles to form PlyA-micelles which are subsequently contacted with mutant PlyB monomers to enable the formation of a PlyAB-micelle complex. The PlyAB-micelle complex is then contacted with a lipid bilayer to allow nanopore formation.

A nanopore of the invention, optionally comprised in a system or devise as described herein above, is advantageously used for single molecule detection of an analyte of interest or particular properties thereof. Properties which may be analyzed include molecular weight, shape and size, net charge, charge distribution and sequence.

Analytes may be added to the trans or cis side of the nanopore. When in use, a nanopore is subjected to an electrical potential such the analyte is electrophoretically and/or electroosmotically translocated through the nanopore. The optimal value (absolute and direction) of the potential depends on the ion-selectivity of the nanopore, characteristics like size, shape and pI of the analyte and whether analytes are captured from the trans or cis side of the nanopore. For instance, for an anion selective pore of the invention, at approximately neutral pH, a positive potential is necessary to capture analytes from the cis side, whereas for trans capture a negative potential is needed. The capture frequency and dwell time within the nanopore for a particular analyte also depend on the absolute value of the potential. Optimization of the electric field for analyte detection using a nanopore should be a routine procedure for someone skilled in the art.

A nanopore of the invention is suitable for detection and analysis of a wide variety of analytes. Such analytes are preferably biological macromolecules, for example a protein, single or double-stranded DNA or RNA. The use of a nanopore for the detection of complexes of biological macromolecules, for example a protein-protein complex or a DNA-protein complex is also possible.

A nanopore according to the invention is particularly advantageously used to study folded proteins. For instance, as shown herein below PlyAB-E1, PlyAB-E2 and PlyAB-R nanopores all display the ability to capture the 24 kDa protein β-casein with a well-defined dwell time and blockage current which should in principle allow for protein identification. Hence, in a preferred embodiment, folded proteins, with a molecular weight above 20 kDa are identified or characterized using a nanopore of the invention.

PlyAB nanopores of the invention are capable of sensing significantly larger folded proteins, or complexes thereof, when compared to known biological nanopores. For instance, tetrameric hemoglobin (64 kDa) could be detected using a PlyAB-E1nanopore and the PlyAB-R nanopore could detect BSA (66.5 kDa), HSA (66.5 kDa) and HTr (76-81 kDa).

Thus, a nanopore according to the invention are particularly advantageously used to study large folded proteins or complexes thereof, preferably with a molecular weight above approximately 40 kDa. In a preferred embodiment a nanopore of the invention is used to characterize a folded protein or protein complex with a molecular weight in the range of 40 to 100 kDa. For instance, such a nanopore is used to sense proteins or complexes thereof with a molecular weight of 41, 42, 45, 50, 53, 57, 59, 62, 64, 66, 67, 71, 73, 76, 81, 85, 90, 92, 93, 96 or 98 kDa. In a particularly preferred embodiment, the molecular weight of the proteinaceous analyte is at least 50 kDa, preferably at least 55 kDa, more preferably at least 60 kDa.

Upon entering the nanopore under influence of the electrophoretic and/or electroosmotic force, an analyte’s interactions with the nanopore depend on its chemical make-up. These interactions give rise to specific residence times of the analyte within the nanopore, known as the dwell time. The analyte’s presence in the nanopore also (partially) blocks the flow of ions through the pore, which can be detected as a change in current. This blockage current (Ires%) again depends on the chemical character of the analyte. As such, chemically distinct analytes may be distinguished using a nanopore of the invention based on their characteristic dwell time and blockage current.

Therefore, in one embodiment, a nanopore of the invention is used to detect and distinguish between different analytes in a mixture, wherein the analytes are biomolecules. Preferably, the nanopore is used to detect and identify different folded proteins from a mixture. For example, the exemplary PlyAB-R nanopore is capable of distinguishing between two human plasma proteins when both are present in a mixture.

Moreover, the exemplary PlyAB-E1 pore was shown to be able to distinguish between hemoglobin species differing only in a point mutation in the two β-subunits of the 64 kDa tetramer. This demonstrates that a nanopore according to the invention has a unique and advantageous sensitivity to comparatively small chemical differences between analytes.

Hence, in one embodiment, a nanopore of the invention is used to detect protein post-translational modifications such as phosphorylation or glycosylation, DNA methylation, binding of ligands to enzymes, and single point mutations in DNA, RNA or a protein. In a preferred embodiment, the nanopore is used to characterize a mixture wherein different species of an analyte, such as a mixture of protein or DNA molecules differing only in their post-translational modifications or in one or more point mutations.

In a further aspect, a nanopore of the invention may be used to analyze a sample for the presence of a biomolecule with mutation or post-translational modification associated with a disease. For instance, the PlyAB-E1 nanopore is suitably used to analyze a sample for the presence of hemoglobin with the E to V mutation associated with sickle cell disease. Hence, in a preferred embodiment, a nanopore of the invention is used to analyze a sample for the presence of a proteinaceous species comprising a post-translational modification or, preferably, one or more point mutations associated with a disease.

Candidate proteins for detection by a nanopore of the invention may vary widely in their pI values. The net charge of the proteinaceous analyte at conditions used for nanopore sensing will affect their interaction with the nanopore interior. For instance, whilst BSA (pI 4.7, net charge -18.5 at pH 7.5) is readily detected by the anion-selective PlyAB-R, it does not enter the cation-selective PlyAB-E2 nanopore. It is anticipated that a cation-selective nanopore is preferred to detect folded proteins with a similarly large positive charge.

Thus, in a preferred embodiment, an anion selective PlyAB nanopore is used to detect proteins with pI values below approximately 5.5, under physiological conditions. In another preferred embodiment, a cation selective PlyAB nanopore is used for detection under physiological conditions of proteins with pI values above approximately 8.

A major advantage of the electrical nature of the signal in nanopore sensing is that it allows analysis of biological samples in real time. Furthermore, since molecules are detected one at a time, nanopores outperform conventional ensemble-based proteomics techniques, like mass spectrometry, when it comes to the detection of low-abundance species and identifying chemical heterogeneity in post translational modifications. These advantages can be further enhanced when a nanopore-based detection setup is combined with high throughput analysis. Hence, the use of a nanopore of the invention, capable of detecting and identifying larger folded protein, in qualitative as well as quantitative proteome analysis is also provided.

### LEGEND TO THE FIGURES

FIG. 1. Structure and size comparison of typical biological nanopores. Side views of cartoon representations of a-hemolysin (a, aHL, PDB ID: 7AHL), cytolysin A (b, ClyA, PDB ID: 2WCD) and two component pleurotolysin nanopores (c, PlyAB, PDB ID: 4V2T [26]) nanopores. The PlyAB nanopore structure was built with homology modelling using the MODELLER software package [27] from the PlyAB Cryo-EM map (PDB ID: 4V2T) with structures of soluble PlyA (PDB ID: 4OEB) / PlyB (PDB ID: 40EJ) monomers [26]. The full structure was minimized for 5 ns with symmetry constrained molecular dynamics flexible fitting (MDFF) to the CryoEM map with NAMD [28].

FIG. 2. Sequence of PlyA. (a) : Amino acid sequence of WT PlyA used to develop mutant PlyA polypeptides. Residues which may be mutated as discussed herein are indicated in bold. The optional His-tag is indicated in italics. (b) DNA sequence encoding PlyA shown in capital letters. The base pairs of restriction sites are shown in italics and underlined.

FIG. 3. Sequence of PlyB. (a) Amino acid sequence of WT PlyB used to develop mutant PlyB polypeptides. Residues which may be mutated as discussed herein are indicated in bold. The optional His-tag including the GSA linker as well as the additional C-terminal residues M and A are indicated in italics and underlined. (b) DNA sequence encoding the PlyB polypeptide of FIG. 3a. The DNA sequence encoding the PlyB core, corresponding to residues 49-523 of Pfam Q5W9E8 are shown in capital letters.

FIG. 4. Gating stability of PlyAB pores. (a) Pores formed of WT PlyA and PlyB-E 1 show spontaneous gating. (b) Similarly, pores comprising WT PlyA and PlyB-E2 regularly open and close spontaneously at -50 mV. However, (c) pores formed of PlyA-S and PlyB-E2 remain open for prolonged times at -50 mV and -150 mV. (d) Pores comprising PlyA-S and PlyB-E1 show a similar stability, indicating that replacement of the cysteines at positions 62 and 94 in PlyA is essential to obtain a stable PlyAB nanopore.

FIG. 5. Protein capture with PlyAB nanopores in 1 M NaCl at pH 7.5. (a) β-casein (24 kDa, pI 5.1) and bovine serum albumin (BSA, 66.5 kDa, pI 4.7) were measured with PlyAB-E2 nanopores. The PlyAB-E2 constriction is negatively charged. β-casein (grey) and BSA (dark grey) were added to the trans and cis side separately and either a positive or negative potential was applied to the trans side. The direction of electrophoretic force (EPF) and electroosmotic flow (EOF) in each experiment are indicated with arrows. (b) β-casein and BSA were measured with PlyAB-R nanopores from both sides. Recordings were collected with a 50 kHz sampling rate and a 10 kHz low-pass Bessel filter.

FIG. 6. Detection of and separation between human albumin (HSA) and transferrin (HTr) in a mixture. (a) Cross-section of PlyAB nanopores (left panel) and surface representations of transferrin (HTr, middle panel) and human albumin (HSA, right panel) are shown to the same scale. (b,c) Individual detection of HSA and HTr, respectively. Left: typical traces under +50 mV applied potential. Right: the heat map of plotting the amplitude standard deviation against the Ires%. (d) The separation of HSA and HTr in a mixture. Recordings were conducted in 300 mM NaCl at pH 7.5, with a 50 kHz sampling and a 10 kHz Bessel filter.

FIG. 7. Discrimination of hemoglobin A and S at pH 7.5. (a) Detection of hemoglobin A (Hemo A). Left: 10 second trace provoked by Hemo A when added in the trans side of PlyAB-E 1 nanopores and measured under +50 mV. Middle: The enlarged trace shows Hemo A signal has two distinctive levels. The deeper level is assigned as level 1 (L₁) and the upper level as level 2 (L₂). Right: Whole trace histogram of Hemo A blockades. (b) Detection of hemoglobin S (Hemo S). (c) The voltage dependence of level 2 percentage for both Hemo A and S. The percentage was calculated from the area of the whole trace histograms. The voltage dependence indicates the two levels within the hemoglobin blockades are most probably reflecting the exchange of dwelling positions inside the pore other than the intrinsic conformation dynamics of hemoglobin. (d) The Ires% of level 2 at difference voltages for Hemo A and S. Ires% was defined as I_{B}/I_{O}*100%, where the I_{B} refers to the blockade currents and Io the open pore currents. Hemo A has a higher Ires comparing with Hemo S in every measured voltage condition, which is likely due to the extra negative charge of glutamic acid residue in Hemo A. More negatively charged Hemo A dwells more in the position less close to the PlyAB nanopore constriction site (level 2), since it encounters a stronger opposite electrophoretic force. The recordings were performed in 300 mM NaCl with a 50 KHz sampling rate and 10 kHz low-pass Bessel filter. (e) Hemo A and S can be detected in a mixture due to their distinct blockage profiles.

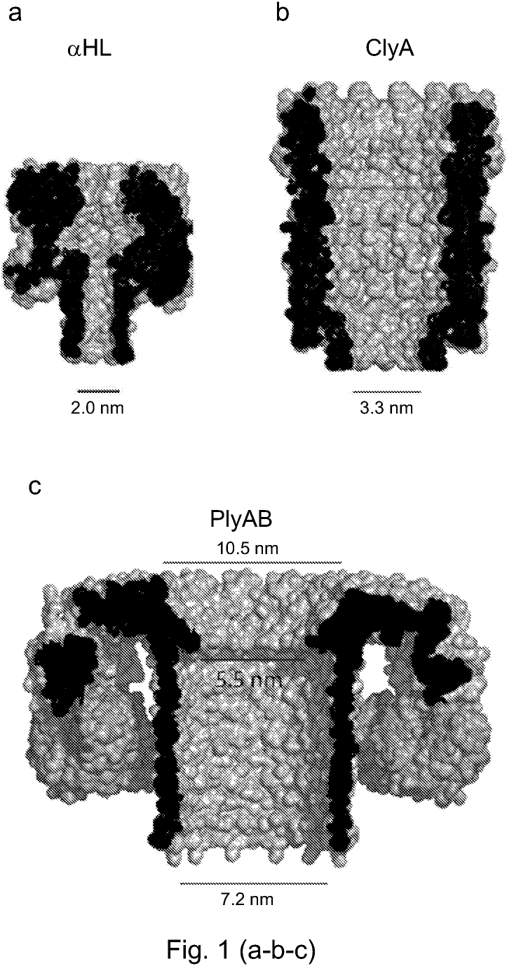

### EXPERIMENTAL SECTION

### Materials and Methods

Synthetic genes and primers were purchased from Integrated DNA technologies (IDT). Enzymes, with the exception of RED polymerase, were acquired from Thermo Scientific and lipids from Avanti Polar Lipids. All other chemicals, plasma proteins, bovine serum albumin (BSA) and RED polymerase were obtained from Sigma Aldrich.

### Example 1: Construction of PlyA and PlyB Mutants

Site-directed mutagenesis was used to produce a PlyA mutant with amino acid substitutions C62S and C94S (PlyA-S) (see Table 2 for primers and Table 3 for an overview of PlyA and PlyB mutants).

TABLE 2

Primers used in site-directed mutagenesis of PlyA and PlyB

| | |
|---|---|
| PlyA-S forward | 5′ GATTAACGCCAGTGGTCGCTC 3′ |
| PlyA-S reverse | 5′ CCCACGGACTATCCCAGTA 3′ |
| PlyB-R forward | 5′ TAATACGACTCACTATAGGG 3′ |
| PlyB-R reverse | 5′ CTTCAGGTCGAAGGCTTCGTCCTCCG 3′ |

TABLE 3

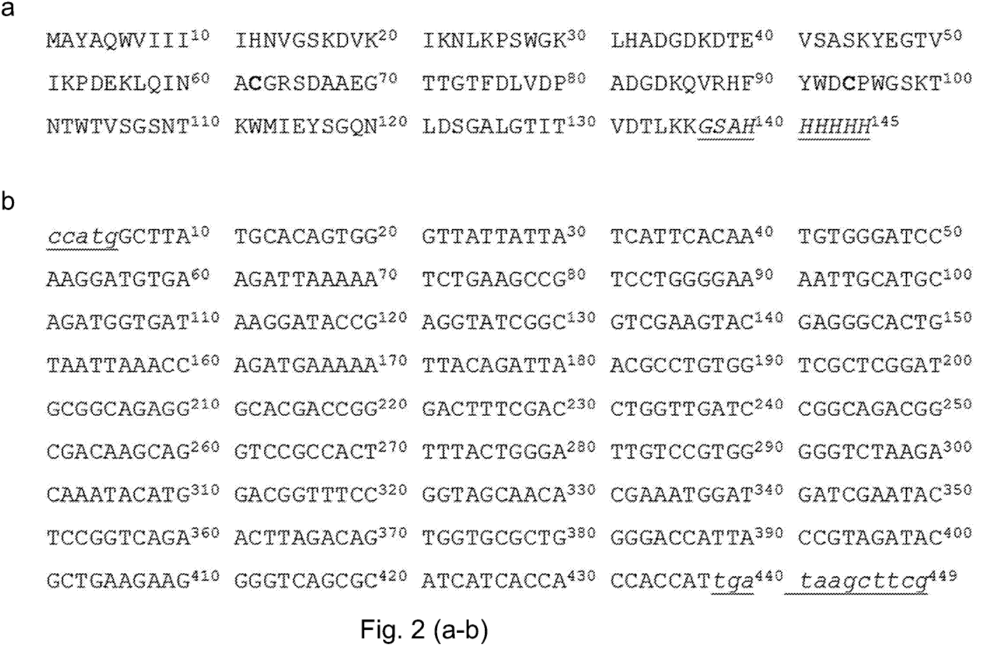

Overview of PlyA and PlyB mutants. Numbering starts from the first residue of FIG. 2a (corresponding to residue 1 of Pfam Q8X1M9) or from residue 49 of Pfam Q5W9E8 respectively for PlyA or PlyB

| | |
|---|---|
| Mutant | Amino acid substitutions |
| PlyA-S | C62S, C94S |
| PlyB-E1 | N72D, A374T, A510V |
| PlyB-E2 | N72D, N153D, G264R, A374T, C487A, A510V |
| PlyB-R | N72D, K301E, E306R, E307R, E316R, A374T, C487A, A510V |

Directed evolution was carried out to provide a PlyB mutant which can be expressed in a soluble way. To that end, a PlyB mutant library was constructed by two steps of PCR amplification. In the first step, ~200 ng wild type PlyB plasmid obtained as described in Example 2 was used as template in a 50 µL PCR reaction system (2 µM T7 promoter primer, 2 µM terminator primer, 25 µL REDTaq ReadyMix).

The PCR protocol started with a pre-denaturing step at 95° C. for 150 seconds, followed by 30 cycles of denaturing at 95° C. for 15 seconds, annealing at 55° C. for 15 seconds and extension at 72° C. for 180 seconds. After the cycles, a final extension step at 72° C. for 300 seconds was to ensure the complete whole gene amplification. REDTaq is a polymerase with relatively low fidelity (2.28 x 10⁻⁵) and 68.4% of the final molecules contain around one base mutation after 30 cycles amplification of a 1 kb DNA template. The PlyB gene contains 1461 base pairs, hence around 1 \~2 mutations per gene could be induced after amplification with REDTaq enzyme without adding extra error-prone enhancement chemicals such as MnCl₂. The first step PCR product (MEGA primer) was purified with QIAquick PCR purification kit and used as primer for second step PCR to amplify the whole plasmid. Therefore, second PCR was performed with high fidelity polymerase Phire hot start II (Finnzymes). 50 µL PCR mix contained 1 µL Phire II, 10 µL 5 x Phire buffer, 0.2 mM dNTPs, 1 µL product from first PCR (200 ng/µL), 1 µL wild type PlyB plasmid and 33 µL PCR water. PCR was conducted with protocol: pre-incubation at 98° C. for 30 seconds, 25 cycles of denaturing and extension (denature: 98° C. for 5 seconds, extension: 72° C. for 240 seconds). The original template plasmid was eliminated by addition of DpnI (1 FDU) and incubation at 37° C. for 1 hour. 1 µL of the treated product was transferred to 50 µL of E. cloni® 10G competent cells (Lucigen) by electroporation.

Cells were grown overnight at 37° C. on agar plate containing 100 µg/mL ampicillin. In next day, all clones were harvest from the plate and used for plasmid preparation. For further activity screen, the plasmid mixture was transferred to E. cloni® EXPRESS BL21 (DE3) cell. At least 190 single clones were picked and inoculated to 96-deep-well plate filled with 400 µL of 2YT media containing 100 µg/mL ampicillin (seed plate). Wild type PlyB was also expressed as control. Clones were grown in plate shaker overnight at 37° C. with gentle shaking. 50 µL of overnight starter of each clone were inoculated into another well in a new plate containing 600 µL of fresh 2YT media with 100 µg/mL ampicillin. Seed plates were stored at 4° C. New culture grew at 37° C. with shaking until the optical density of 600 nm was around 0.6 (2 \~3 hours) and 0.5 mM final concentration of IPTG was added to each well to induce overnight expression at 25° C. Cell culture was spun down in the second day with 2000 xg for 30 minutes and stored at -80° C. overnight after discarding the supernatant. After overnight freezing, 300 µL lysis buffer (150 mM NaCl, 15 mM Tris pH 7.5, 1 mM MgCl₂, 0.2 mg/ml lysozyme, one cOmplete™ Protease Inhibitor Cocktail tablet per 30 mL, 0.05 units/ml DNase and 0.1% 2-Mercaptoethanol) were added to each well to resuspend the pellet. Plates were kept shaking for 3 hours at room temperature for the cell lysis. Then, suspension was centrifuged down with 2000 ×g for 30 minutes and the soluble expressed PlyB monomer protein of each clone should be in supernatant.

In order to test the expression level and toxicity, the hemolytic activity of each clone was tested using a hemolytic assay. The hemolytic assay was performed as follows. Sheep blood cell suspension (Thermo Scientific) was pre-washed with SDEX buffer (150 mM NaCl, 15 mM Tris, pH 7.5) until the supernatant was clear. The erythrocytes were then resuspended in the same buffer to a concentration corresponding to an OD 650 nm around 0.8. Washed sheep erythrocyte cells were first supplemented with 0.01 mg/mL wild type PlyA monomer (final concentration) and kept at room temperature for 10 minutes. Subsequently, 100 µL of PlyA-erythrocyte mixture was transferred to a well on transparent 96-well plate and 5 µL lysate supernatant from PlyB clone was added. The hemolytic activity was measured by monitoring the decrease in OD650 using a Multiskan GO microplate Spectrophotometer (Thermofisher).

Highly hemolytically active clones were isolated and used as a template for the subsequent round of directed evolution.

After three rounds of directed evolution, the PlyB-E1 mutant, comprising the amino acid substitutions N72D, A374T and A510V in the soluble part and showing desirable degrees of soluble expression and hemolytic activity, was identified. After another two rounds of directed evolution, the PlyB-E2 mutant, further comprising the amino acid substitutions N153D, G264R and C487A was identified.

Mutant PlyB-E1 was further optimized to create an anion selective nanopore. Specifically, the amino acid substitutions E306R, E307R, E316R and C487A were introduced using site-directed mutagenesis (see Table 2 for primers used in site-directed mutagenesis).

As the resulting mutant was not sufficiently soluble, another round of directed evolution was performed to obtain the PlyB-R mutant comprising the following amino acid substitutions: N72D, K301E, E306R, E307R, E316R, A374T, C487A and A510V.

### Example 2: Expression and Purification of PlyA and PlyB Monomers

To allow for cloning, a NcoI restriction site was introduced at the beginning of the DNA sequence (5′ end) corresponding to residues 49-523 of WtPlyB or to residues 3-149 of WtPlyA. To maintain the reading frame an additional two bases were inserted after the Nco I site, resulting in an additional alanine residue after the starting methionine. For PlyA this means the polypeptide sequence corresponds to the WtPlyA sequence (Pfam Q8X1M9). For purification purposes, at the C-terminus of the PlyA or PlyB polypeptide, a His6 affinity tag was attached via a flexible glycine-serine-alanine linker and the open reading frame was terminated by two consecutive stop codons, followed by a Hind III restriction site (3′ end). Synthetic genes of pleurotolysin A and B (PlyA, PlyB) or mutants thereof were digested by enzyme recognizing the NcoI and HindIII restriction sites at the 5′ and 3′ ends, and ligated to an expression pT7-SC 1 plasmid predigested with same enzymes.

E.cloni® EXPRESS BL21 (DE3) cells were transformed with the pT7-SC1 plasmid containing the PlyA or PlyB gene by electroporation. Transformants were selected after overnight growth at 37° C. on LB agar plates supplemented with 100 µg/ml ampicillin. Clones were harvested from plate and inoculated into 200 mL fresh sterile 2YT media, supplemented with 100 µg/mL ampicillin. Cell culture were grown at 37° C. with 220 rpm shaking until reaching an OD₆₀₀ nm of approximately 0.6. The expression of PlyA or B was then induced by adding IPTG to a final concentration of 0.5 mM.

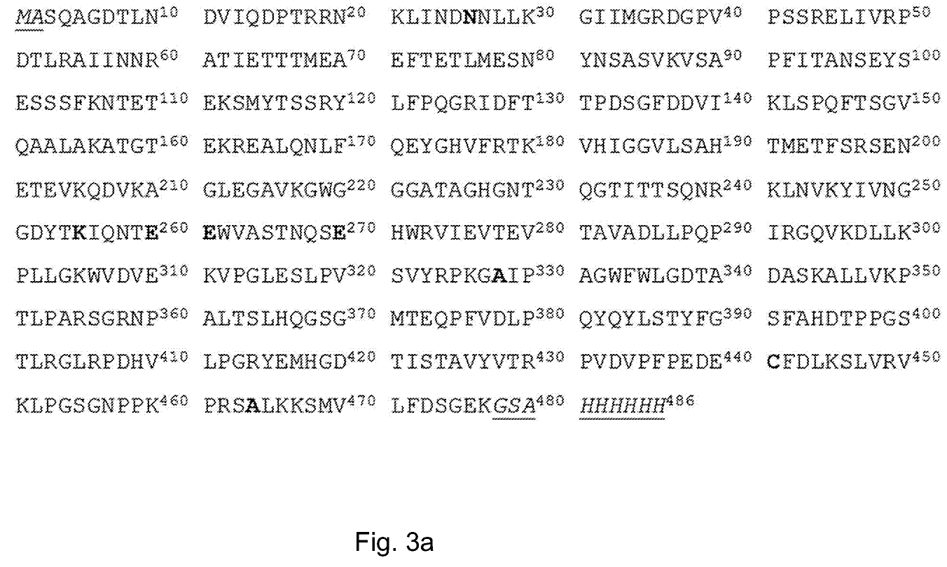

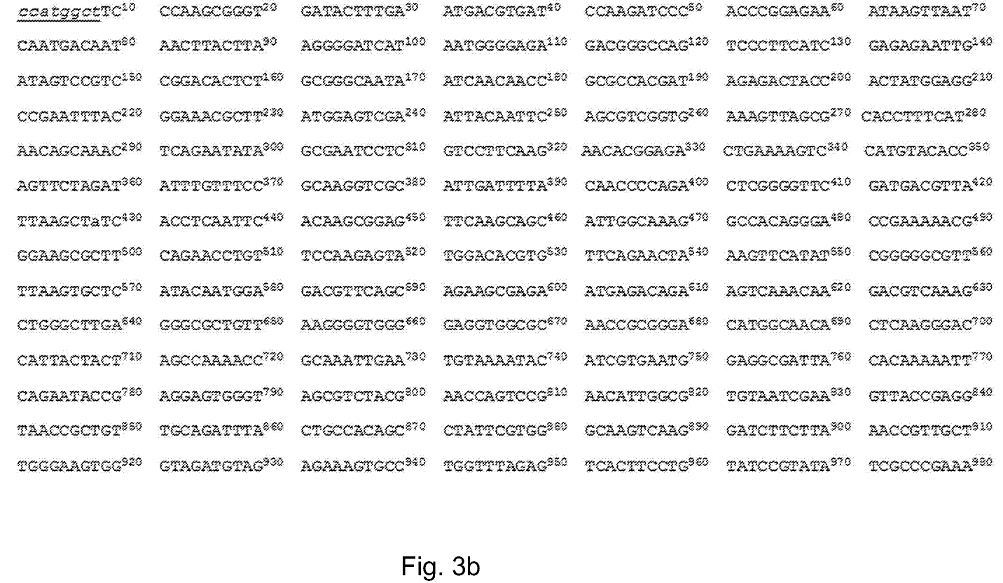

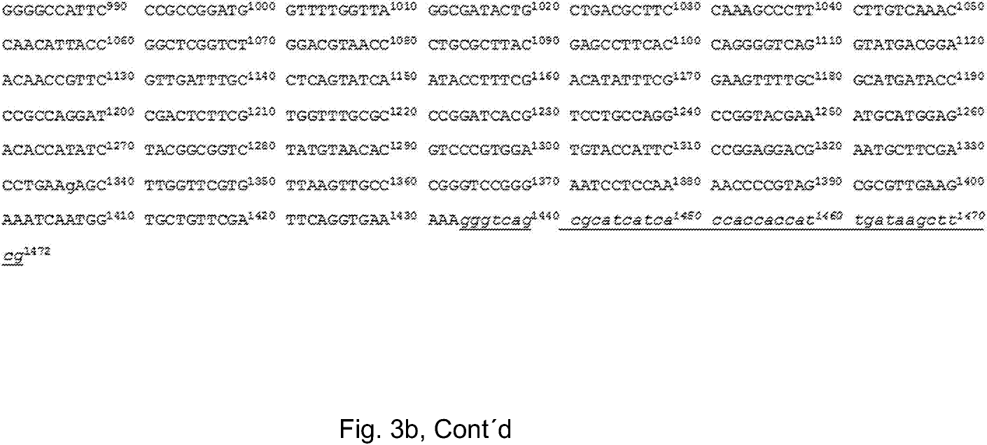

Growth was continued overnight at 25° C. with 220 rpm shaking. Cells were harvested by centrifugation at 2000 ×g for 30 minutes at 4° C. and pellets were stored at -80° C. Pellets derived from 100 ml of bacterial culture were used for protein purification by first resuspending in 30 mL lysis buffer (150 mM NaCl, 15 mM Tris, 1 mM MgCl2, 0.2 mg/ml lysozyme, one cOmplete™ Protease Inhibitor Cocktail tablet and 0.05 units/ml DNase, pH 7.5) and subjected to vigorous shaking for 1 hour at room temperature. Cell lysate was sonicated for 2 minutes (duty cycle 10%, output control 3 using a Branson Sonifier 450) and centrifuged at 4° C. (5400 ×g for 30 minutes). 100 µL (solution volume) of Ni-NTA beads (Qiagen) were washed with 1 mL buffer (150 mM NaCl, 15 mM Tris, 10 mM imidazole, pH 7.5) for 3 times and then the beads were added to the lysate suspension and left to incubate at room temperature for 1 hour under gentle mixing. Resins were then spun down at low speed (2000 ×g) for 5 minutes at 4° C. Subsequently, the beads were loaded to a Micro Bio-Spin column (Bio-Rad). The Ni-NTA beads were washed with 10 mL wash buffer (150 mM NaCl, 15 mM Tris, 10 mM imidazole, pH 7.5) and protein was eluded with 150 µL elution buffer (150 mM NaCl, 15 mM Tris, 300 mM imidazole, pH 7.5). Protein monomers were stored at 4° C. For the purification of PlyA and PlyB monomers which contained cysteine, all buffers mentioned above were supplemented with 0.1% 2-mercaptoethanol. The sequences of WT PlyA and WT PlyB monomers used are shown in FIG. 2a and FIG. 3a, respectively.

### Example 3: Preparation of a System Comprising an Engineered PlyAB Nanopore in a Lipid Membrane

The genetically engineered PlyA and PlyB polypeptides described above are advantageously used in a nanopore according to the invention. A system comprising such a PlyAB nanopore may be prepared by first reconstituting the protein monomers in liposomes, followed by contacting the lipoprotein mixtures with a suitable lipid bilayer.

Cholesterol:sphingomyelin liposomes were prepared by dissolving 25 mg each of cholesterol and sphingomyelin (1:1 mixture) in 5 mL pentane supplemented with with 0.5% v/v ethanol (to help dissolving sphingomyelin) and transferred to a round bottom flask. The solvent was evaporated while slowly rotating the flask in order to deposit a lipid film on the walls. After deposition of the lipid film, the flask was kept open for 30 min at room temperature to allow complete evaporation of the solvent. The lipid film was then resuspended in 5 ml SDEX buffer (150 mM NaCl, 15 mM Tris, pH 7.5) using a bath sonicator for approximately 5 minutes at ambient temperatures. Obtained liposomes may be stored at -20° C. at a final total lipid concentration of 10 mg/ml.

In order for PlyAB nanopore to assemble correctly, reconstitution of the protein monomers into the liposomes is a two-step process. First, PlyA monomer was mixed with the cholesterol-sphingomyelin liposomes in a 1:10 mass ratio and kept at ambient temperature for 10 minutes. Then, PlyB monomer was added to the lipoprotein mixture to a final PlyA:PlyB:liposome mass ratio of 1:1:10. The resulting mixture was kept at room temperature for 2 hours. The PlyAB lipoprotein mixture may be stored at 4° C.

Subsequently, the PlyAB lipoprotein mixture is contacted with a planar lipid bilayer to obtain a system according to the invention. In this Example, the lipid bilayer is included in an electrophysiology chamber. The electrophysiology chamber was separated by a 25 µm-thick polytetrafluoroethylene film (Goodfellow Cambridge Limited) into two compartments (cis and trans). There was 100 µm diameter hole in the center of the film, pretreated with approximately 5 µl of 5% v/v hexadecane in pentane. Both compartments were filled with 500 µl of buffer and a planar bilayer was formed by addition of 10 µL of 10 mg/ml 1,2-diphytanoyl-sn-glycero-3-phosphocholine (DPhPC) in pentane to both chambers. Addition of 0.5 µl of PlyAB lipoprotein (total amount of protein 1 µg) to the cis compartment was typically sufficient to obtain a system comprising a single nanopore assembled into the DPhPC bilayer.

### Example 4: Gating Stability of PlyAB Pores

For electrophysiological experiments to be reliable, a nanopore according to the invention should not display significant opening and closing in the absence of analytes. In other words, such a nanopore should have sufficient gating stability.

Gating stability was evaluated for different combinations of PlyA and PlyB mutants by preparing a system as described in Example 3 and applying a transmembrane potential. Electrical signals from planar bilayer recordings were amplified using a Axopatch 200B patch clamp amplifier (Axon Instruments) and digitized with a Digidata 1440 A/D converter (Axon Instruments). Ag/AgCl electrodes connected the two compartments of the electrophysiology chamber to the patch clamp amplifier and the electrical signal digitizer, with the ground electrode connected to the cis compartment and the working electrode to the trans compartment. Data was recorded using the Clampex 10.4 software (Molecular Devices) and subsequent analysis was carried out with Clampfit software (Molecular Devices). The dwell time, inter-event time, blocked pore current value (I_{B}) of each event, and open pore current value (Iₒ) were determined by the “single channel search” function in Clampfit. Residual current values (Iᵣₑₛ) were calculated from I_{B} and Io as Iᵣₑₛ=I_{B}/I_{O}*100% and used to describe the blockade amplitude for each event. Average dwell time and inter-event time was calculated by fitting single exponentials to histograms of cumulative distribution of all events.

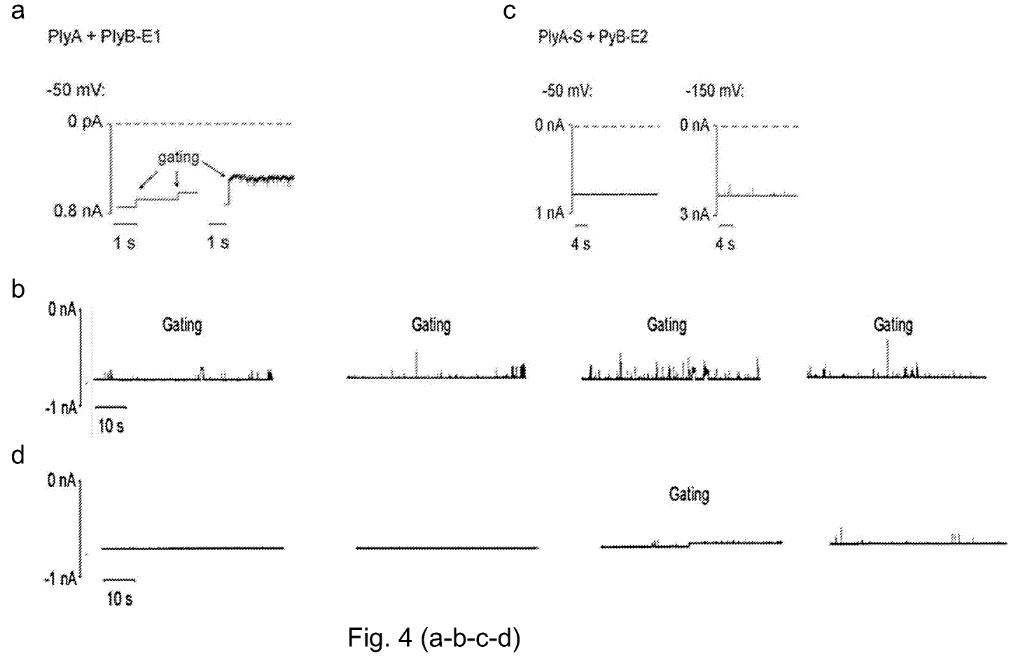

Pores formed of WT PlyA and PlyB-E 1 monomers (PlyAB-WTE1 nanopores, see Table 4 for naming convention of the PlyAB nanopores characterized herein) showed significant spontaneous gating (i.e. opening and closing of the pore) as exemplified by the pore current trace in FIG. 4a. Similarly, nanopores formed of WT PlyA and PlyB-E2 monomers (PlyAB-WTE2 nanopores) also displayed gating in the absence of analytes (FIG. 4b).

TABLE 4

Overview of PlyAB pores

| | | |
|---|---|---|
| Pore | PlyA and B mutants | Conductance (nS) |
| PlyAB-WTE1 | PlyA and PlyB-E1 | Not measured, spontaneous gating |
| PlyAB-WTE2 | PlyA and PlyB-E2 | Not measured, spontaneous gating |
| PlyAB-E1 | PlyA-S and PlyB-E1 | 14.9±0.2 (1 M NaCl, -50 mV, n=46) |
| PlyAB-E2 | PlyA-S and PlyB-E2 | 15.4±0.3 (1 M NaCl, -50 mV, n=53) |
| PlyAB-R | PlyA-S and PlyB-R | 15.3+0.8 (1 M NaCl, +50 mV, n=112) 5.4+0.2 (0.3 M NaCl, +50 mV, n=44) |

Conversely, PlyAB-E1 (FIG. 4d) or PlyAB-E2 (FIG. 4c) nanopores comprising PlyA subunits wherein cysteines 62 and 94 have been replaced with serine, in combination with either PlyB-E1 or PlyB-E2 are surprisingly stable. For example, the PlyAB-E2 nanopore routinely remained open at an applied potential of -150 mV for tens of seconds (FIG. 4c).

It is therefore hypothesized that the electrical stability in engineered PlyAB nanopores is most likely inferred by removal of the cysteine residues in PlyA. These residues are located at the interface with the lipid membrane and are known to be involved in lipid binding. Therefore, a nanopore according to the invention preferably comprises PlyA monomers wherein one, or preferably both, C62 and C94 have been replaced by another amino acid, in this case serine.

### Example 5: Ion-Selectivity of PlyAB Pores

In nanopores, the degree of ion-selectivity and the direction of the electroosmotic flow are often correlated, as they both result from the interaction of the electrolyte ions with the fixed charges on the nanopore walls. As such, ion-selectivity is a key characteristic of a nanopore and will affect its ability to sense particular analytes.

The ion-selectivity of a nanopore can be derived from its reversal potential using the Goldman-Hodgkin-Katz equation:

$$\frac{{P}_{N a} +}{{P}_{{\text{Cl}}^{-}}} = \frac{{\left[{a}_{\text{Cl}} -\right]}_{t r a n s} - {\left[{a}_{\text{Cl}} -\right]}_{c i s} \text{exp} \left(\frac{{V}_{r} F}{R T}\right)}{{\left[{a}_{\text{Na}} +\right]}_{t r a n s} \text{exp} \left(\frac{V {r}^{F}}{R T}\right) - {\left[{a}_{\text{Na}} +\right]}_{c i s}} ,$$

where R is the gas constant, T is the temperature, F is the Faraday’s constant, and Vᵣ is the reversal potential measured using asymmetric salt conditions.

To assess ion-selectivity, a single PlyAB nanopore in symmetric salt conditions was prepared by adding 400 µl of 1 M NaCl, 15 mM Tris, pH 7.5 buffer in both compartments and balancing the electrodes. Then, 400 µL of 3 M NaCl was added to the cis chamber and an identical volume of salt free buffer was added into the trans chamber to create a salt gradient (cis:trans = 500 mM : 2 M). The solution in both chambers was mixed gently and current-voltage (I-V) curves were collected to obtain the reversal potentials.

The reversal potentials measured for nanopores comprising PlyA-S and PlyB-E2 (PlyAB-E2) or PlyB-E1 (PlyAB-E1) in planar lipid bilayers indicate they are slightly cation-selective (see Table 5). This most likely results from the relatively high density of negatively charged amino acids in the constriction zones of these pores.

TABLE 5

Reversal potential and ion-selectivity of PlyAB nanopores

| Pore | Reversal potential (mV) | P_{Na}+/P_{CL}- |
|---|---|---|
| PlyAB-E1 | 1.24±0.2 | 1.08±0.02 |
| PlyAB-E2 | 1.1±0.28 | 1.07±0.02 |
| PlyAB-R | -0.9±0.57 | 0.94±0.04 |

Replacement of E306, E307 and E316 with arginine, as was done in the PlyB-R mutant, results in a similarly high density of positive charges in the constriction zone. Nanopores formed by PlyA-S and PlyB-R (PlyAB-R) displayed a slightly asymmetric conductance, with higher currents at positive applied bias. The current asymmetry was more accentuated at lower ionic strengths. PlyAB-R pores were weakly anion-selective (Table 5). Thus, whilst PlyAB-E1 and PlyAB-E2 nanopores are cation-selective, this ion-selectivity can be tuned, and even reversed, by replacing one or more of the negatively charged glutamic acid residues 306, 307 and 316 in the constriction zone with positively charged amino acids as in the anion-selective PlyAB-R nanopore.

### Example 6: Protein Capture With PlyAB Nanopores

The ability of various engineered PlyAB nanopores to capture and analyse proteins was tested in electrophysiology experiments using two differently sized proteins: β-casein (24 kDa, pI = 5.1, net charge -5.8) and bovine serum albumin (BSA, 66.5 kDa, pI = 4.7, net charge -18.5). Protein capture was tested in 1 M NaCl and pH 7.5.

TABLE 6

Protein capture with PlyAB nanopores

| Pore | Protein | Side | Ires (%) | Dwell time (ms) | Capture Frequency (S⁻¹µM⁻¹) |
|---|---|---|---|---|---|
| PlyAB-E1 | β-casein | Cis | No capture | - | - |
| PlyAB-E1 | β-casein | Trans | 84.9±1.6 | 8.1±1.3 | 166.5±3.4 |
| PlyAB-E2 | β-casein | Cis | No capture | - | - |
| PlyAB-E2 | β-casein | Trans | 84.2±0.1 | 25.0±6.3 | 174.5±120.9 |
| PlyAB-E2 | BSA | Cis | No capture | - | - |
| PlyAB-E2 | BSA | Trans | No capture | - | - |
| PlyAB-R | β-casein | Cis | 93.9±1.1 | 2.8±1.7 | 135.1±95.9 |
| PlyAB-R | β-casein | Trans | 93.8±0.5 | 1.6±0.1 | 50.6±2.6 |
| PlyAB-R | BSA | Cis | 38.4±0.1% | 177.1±138.6 | 527.7±296.1 |
| PlyAB-R | BSA | Trans | 40.9±1.4% | 22.0±13.6 | 365.6±58.9 |

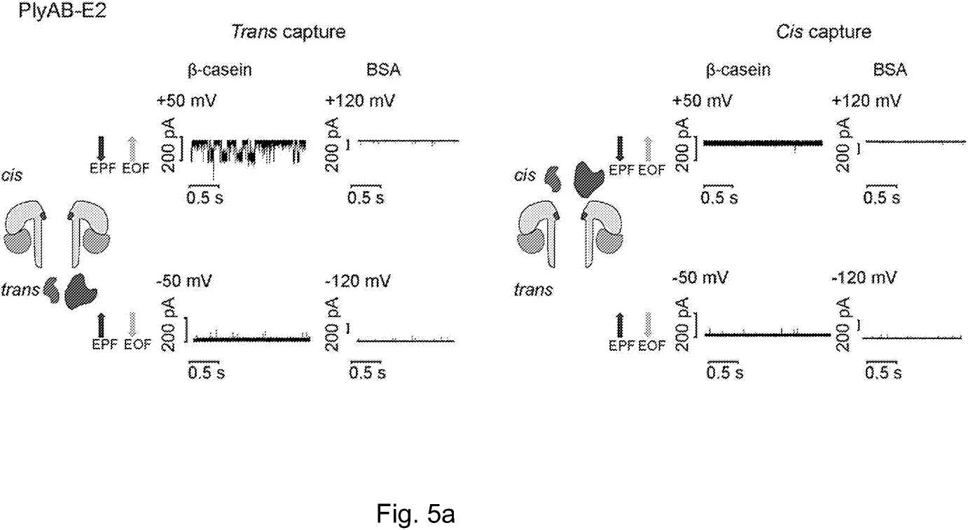

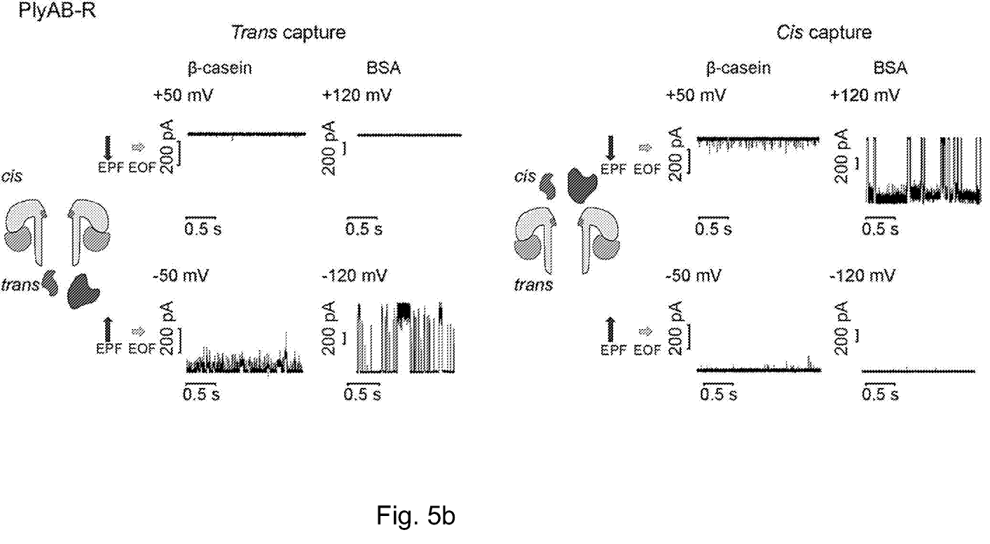

Protein blockades to PlyAB-E1and PlyAB-E2 nanopores were only observed with β-casein, and only when the protein was added to the trans side under positive applied potentials (trans) (FIG. 5a and Table 6). Under these conditions, the electroosmotic force (EOF) promotes the entry of the protein, while the electrophoretic force (EF) acts in the opposite direction. It is likely that for B-casein, which is smaller than the nanopore constriction, the competition between the electrophoretic and electroosmotic forces allows the trapping of the protein within the lumen of the nanopore. Most likely, the large electrophoretic (net charge -18.5 at pH 7.5) and entropic barrier (66.5 kDa) of BSA compared to β-casein (net charge -5.8 at pH 7.5 and 24 kDa) prevented BSA entry into the cation-selective PlyAB-E1and PlyAB-E2 nanopores.

When using PlyAB-R (FIG. 5b) both β-casein and BSA blockades were observed, and proteins could be captured from either sides of the nanopore according to the direction of the EF. During trans capture, β-casein blockades of PlyAB-R were shorter in duration than blockades of PlyAB-E2 or PlyA-E1 (Table 6), suggesting that the competition between electrophoretic and electroosmotic forces is important for obtaining long residence times inside the nanopore. BSA, which could not be captured by the cation-selective PlyAB nanopores tested herein, entered PyAB-R nanopores, although only at relatively high applied potential (e.g. > +100 mV, FIG. 5b). Interestingly, BSA residual currents for cis and trans captures were similar (Table 6), suggesting that in both scenarios the protein is lodged within the same region of the nanopore, presumably the narrower β-barrel vestibule. Most likely, BSA capture is enabled by the reduced electroosmotic flow, which opposes electrophoretic capture of the protein. Electroosmotic vortices in PlyAB-R are also likely to play a role in trapping BSA inside the nanopore. Notably, we found that cis capture was more efficient than trans capture (Table 6), most likely reflecting the larger capture radius of the cis side. Finally, the duration of BSA blockade was different depending on the direction of entry, suggesting that the interaction between the constriction and the protein during translocation plays a role. It can be inferred from the above that an anion-selective PlyAB nanopore, for instance PlyAB-R, is preferred over a cation-selective PlyAB, like PlyAB-E1 or PlyAB-E2, for the detection of proteins with a large negative net charge.

### Example 7: Detection of and Discrimination Between Human Plasma Proteins

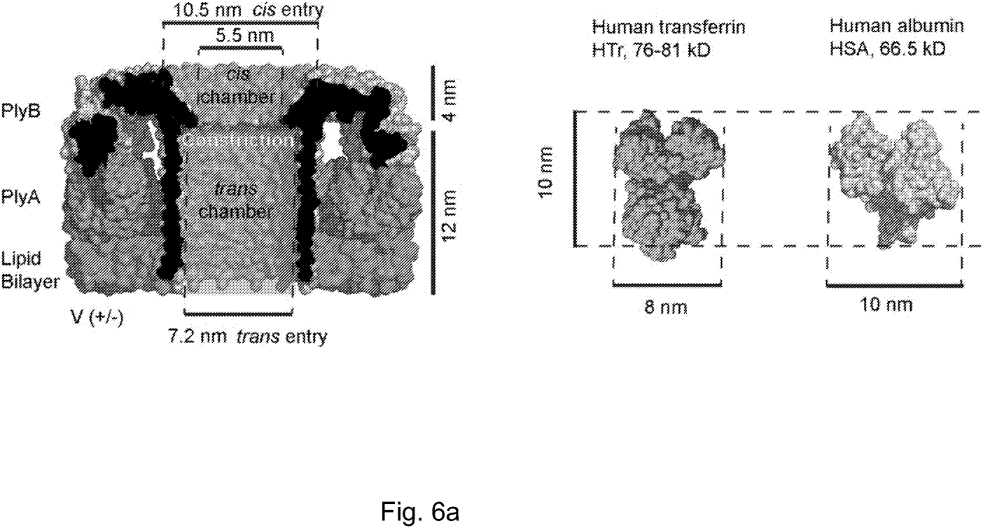

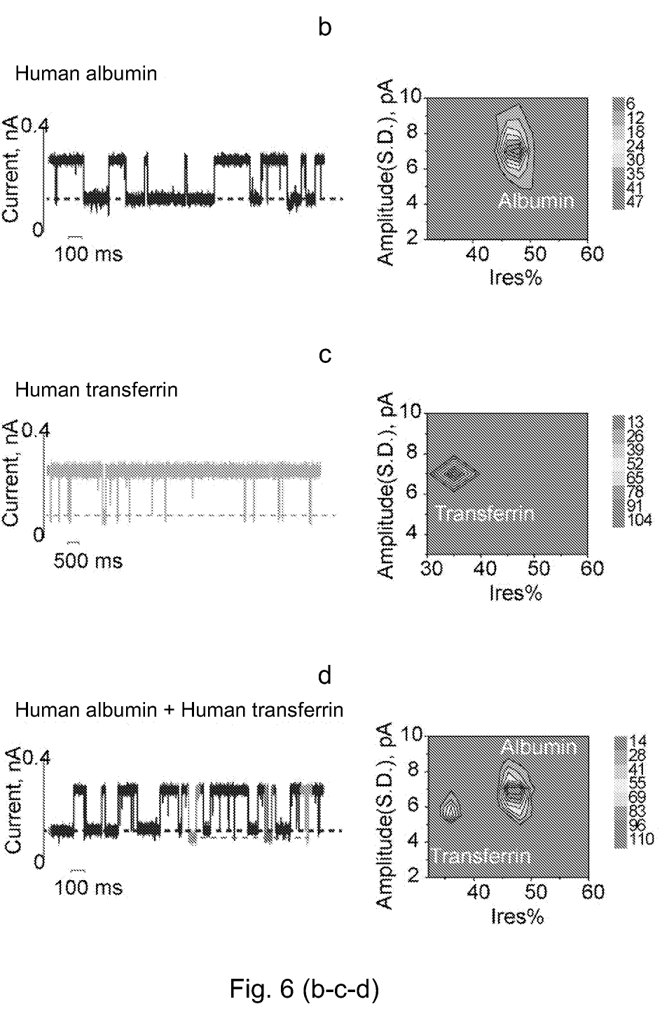

One of the aims of the inventors was to develop a nanopore capable of sensing folded proteins with a molecular weight over approximately 40 kDa. To verify if the engineered PlyAB nanopores of the invention could meet this aim, the ability of PlyAB-R nanopores to detect two human plasma proteins: human albumin (HSA, 66.5 kDa, pI = 4.7) and human transferrin (HTr, 76-81 kDa, pI=5.8) was tested. HSA accounts for 55% of blood protein and is an important transporter for many substrates like lipids, steroid hormones and drugs. HTr is a glycoprotein that controls the level of iron in biological fluids (FIG. 6a).

Since the electroosmotic flow influences the capture and the residence of proteins inside the nanopore, we used 300 mM NaCl solutions, which are expected to increase the relative force of the electroosmotic flow and improve the detection of the plasma proteins. Blockades were characterized by measuring the Ires%, which is defined as the ionic current associated with a protein-blocked pore I_{B} divided by the open pore current Iₒ percent.

Homogeneous and well-defined single current blockades were observed with PyAB-R nanopores for both HSA and HTr (FIGS. 6b,c, Table 7) from the cis side. Higher applied potentials were required to observe blockades when HTr and HSA were added to the trans side (Table 7), reflecting the higher entropic barrier for trans entry compared to the cis entry.

TABLE 7

Human transferrin (HTr) and human serum albumin (HSA) measurements with PlyAB-R nanopores in 300 mM NaCl, pH 7.5

| Protein | Condition | Side | Ires (%) | Dwell time (ms) | Capture Frequency (s⁻ ¹µM⁻¹) |
|---|---|---|---|---|---|
| HTr | +50 mV | Cis | 33.5±1.1 | 30.3±5.4 | 11.1±6.4 |
| HTr | -200 mV | Trans | 37.7±0.1 | 5.4±0.8 | 157.3+136.6 |
| HSA | +50 mV | Cis | 46.3±0.9 | 118.5±43.0 | 54.2±25.8 |
| HSA | -200 mV | Trans | 41.3±0.1 | 4.1±0.7 | 5853.5±1619.5 |

Under +50 mV, HSA and HTr added to the cis side showed distinct Ires% (46.3±0.9% and 33.5±1.1%, respectively), which reflected the different volumes excluded by the two proteins.

Notably, the translocation of proteins in solid-state nanopores with dimensions similar to that of PlyAB is generally fast (typically microseconds) compared to the trapping times observed here for HSA and HTr (118.5 ± 43.0 ms and 30.3±5.4 ms for HSA and HTr at +50 mV, respectively) [29], which complicates protein identification [30, 31, 32, 33].

Moreover, as blockages of a PlyAB-R nanopore with HSA or HTr have characteristic signatures, HSA and HTr could be identified from a mixture on the basis of individual blockades (FIG. 6d).

Thus, a genetically engineered PlyAB nanopore of the invention is capable to detect large folded proteins. Importantly, such a nanopore can also be used to distinguish between different proteins of roughly similar size. Note that the proteins used in this example, particularly HSA, have a relative large negative net charge and hence the PlyAB-R pore is preferred for this application.

### Example 8 Discrimination Between Hemoglobin A and S

For proteomics applications, it is particularly desirable if a nanopore is not only capable to distinguish between two different protein species of similar molecular weight, but also if it can detect small modifications like post-translational modifications and single point mutations. Therefore, the ability of a PlyAB nanopore to distinguish between two different versions of hemoglobin was assessed.

Hemoglobin is a tetramer, comprising two α-subunits and two β-subunits, with a combined molecular weight of approximately 64 kDa (pI=6.8). Human hemoglobin (Hemo A) comprises a glutamic acid at position 6 of the β subunit. Mutation of this residue to valine (Hemo S) is associated with sickle cell disease.

Hemo A or Hemo S was added to the trans side of PlyAB-E1 nanopores and measured under +50 mV. The recordings were performed in 300 mM NaCl with a 50 KHz sampling rate and 10 kHz low-pass Bessel filter.

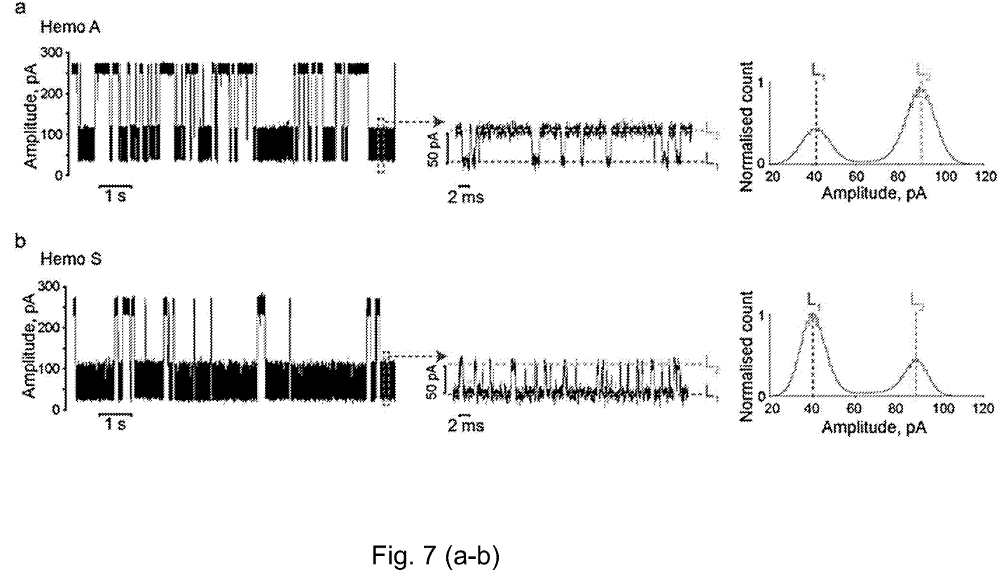

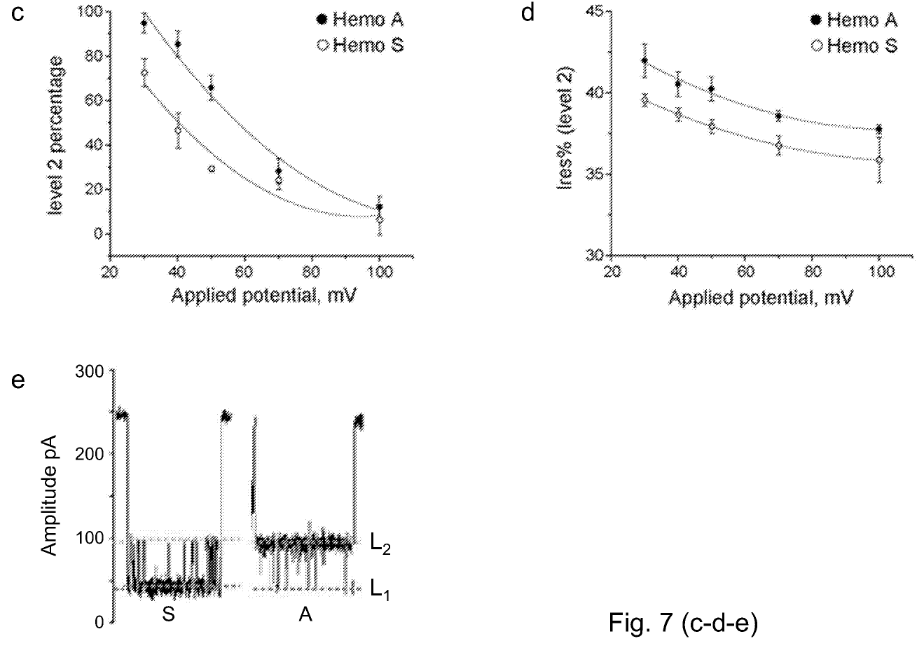

Both Hemo A and Hemo S show two distinct blockade levels (L1 and L2) (FIG. 7a and b) of the PlyAB-E 1 nanopore. The occupancy of these two levels depends on the applied potential (FIG. 7c), indicating that they reflect different dwelling position inside the pore.

Depending on the applied potential, the more negatively charged Hemo A has a preference for the L2 dwelling position which is further away from the nanopore constriction site. Hemo S on the other hand favors the L1 dwelling position. Moreover, at any applied potential measured , Hemo A has a higher Ires% of L2 than Hemo S (FIG. 7d). These distinct blockage signatures allow separation of sickle cell hemoglobin from normal hemoglobin when measured in a mixture (FIG. 7e).

Thus, the PlyAB-E1 nanopore is capable of distinguishing between protein complexes of roughly 64 kDa which differ only in a point mutation in two of the four subunits. This suggests that nanopores of the invention may be advantageously used to detect small differences between large biomolecules.

### References

G. Baaken, “High-resolution size-discrimination of single nonionic synthetic polymers with a highly charged biological nanopore,” ACS Nano, vol. 9, pp. 6443-6449, 2015.

J. W. F. Robertson, “Single-molecules mass spectrometry in solution using a solitary nanopore,” Proc. Natl. Acad. Sci., vol. 104, pp. 8207-8211, 2007.

O. V. Krasilnikov, C. G. Rodrigues and S. M. Bezrukov, “Single polymer molecules in a protein nanopore in the limit of a strong polymer-pore attraction.,” Phys. Rev. Lett., vol. 97, pp. 1-4, 2006.

B. Division, C. Biology and S. Cruz, “Characterization of individual polynucleotide molecules using a membrane channel,” Proc. Natl. Acad. Sci., vol. 93, pp. 13770-13773, 1996.

S. Brenner, “Sequence-specific detection of individual DNA polymerase complexes in real time using a nanopore,” Nat. Nanotechnology, vol. 2, pp. 718-724, 2007.

G. Maglia, M. R. Restrepo, E. Mikhailova and H. Bayley, “Enhanced translocation of single DNA molecules through alpha-hemolysin nanopores by manipulation of internal charge,” Proc. Natl. Acad. Sci., vol. 105, pp. 19720-19725, 2008.

I. M. Derrington, “Nanopore DNA sequencing with MspA,” Proc. Natl. Acad. Sci., vol. 107, pp. 16060-16065, 2010.

J. Clarke, “Continuous base identification for single-molecule nanopore DNA sequencing,” Nat. Nanotechnology, vol. 4, pp. 265-270, 2009.

M. Pastoriza-Gallego, “Dynamics of unfolded protein translocation through an aerolysin pore,” J. Am. Chem. Soc., vol. 133, pp. 2923-2931, 2011.

M. Pastoriza-Gallego, “Evidence of unfolded protein translocation through a protein nanopore,” ACS Nano, vol. 8, pp. 11350-11360, 2014.

G. Huang, A. Voet and G. Maglia, “FraC nanopores with adjustable diameter identify the mass of opposite-charge peptide with 44 dalton resolution,” Nat. Commun., vol. 10, pp. 1-10, 2019.

L. Restrepo-Pérez, “Resolving chemical modifications to a single amino acid within a peptide using a biological nanopore,” ACS Nano, 2019.

L. Restrepo-Pérez, C. H. Wong, G. Maglia, C. Dekker and C. Joo, “Label-free detection of post-translational modifications with a nanopore,” Nano Lett., vol. 19, pp. 7957-7964, 2019.

Z. Ji and P. Guo, “Channel from bacterial virus T7 DNA packaging motor for the differentiation of peptides composed of a mixture of acidic and basic amino acids,” Biomaterials, vol. 214, p. 119222, 2019.

F. Piguet, “Identification of single amino acid differences in uniformly charged homopolymeric peptides with aerolysin nanopore,” Nat. Commun., vol. 9, p. 966, 2018.

G. Huang, K. Willems, M. Soskine, C. Wloka and G. Maglia, “Electro-osmotic capture and ionic discrimination of peptide and protein biomarkers with FraC nanopores,” Nat. Commun. , vol. 8, pp. 1-13, 2017.

A. E. Chavis, “Single molecule nanopore spectrometry for peptide detection,” ACS Sensors, vol. 2, pp. 1319-1328, 2017.

A. Asandei, “Electroosmotic trap against the electrophoretic force near a protein nanopore reveals peptide dynamics during capture and translocation,” ACS Appl. Mater. Interfaces, vol. 8, pp. 13166-13179, 2016.

L. Movileanu, J. P. Schmitt, J. M. Scholtz and H. Bayley, “Interactions of peptides with a protein pore,” Biophys. J. , vol. 89, pp. 1030-1045, 2005.

A. J. Boersma and H. Bayley, “Continuous stochastic detection of amino acid enantiomers with a protein nanopore,” Angew. Chem. Int. Ed. , vol. 51, pp. 9606-9609, 2012.

Z. Tang, “Surface modification of solid-state nanopores for sticky-free translocation of single-stranded DNA,” Small, vol. 10, pp. 4332-4339, 2014.

M. Wanunu and A. Meller, “Chemically modified solid-state nanopores,” Nano Lett. , vol. 9, pp. 1580-1585, 2007.

L. Kapinos, “Single-molecule transport across an individual biomimetic nuclear pore complex,” Nat. Nanotechnology, vol. 6, pp. 433-438, 2011.

H. Watanabe, “Analysis of pore formation and protein translocation using large biological nanopores,” Anal. Chem., vol. 89, pp. 11269-11277, 2017.

C. F. Reboul, J. C. Whisstock and M. A. Dunstone, “Giant MACPF/CDC pore forming toxins: a class of their own,” Biochim. Biophys. Acta - Biomembr., vol. 1858, pp. 475-486, 2016.

N. Lukoyanova, “Conformational changes during pore formation by the perforin-related protein pleurotolysin,” PLoS Biol., vol. 13, pp. 1-15, 2015.

A. Sali and T. L. Blundell, “Comparative protein modelling by satisfaction of spatial restraints,” J. Mol. Biol., vol. 234, pp. 779-815, 1993.

J. C. Phillips, “Scalable molecular dynamics with NAMD,” J. Comput. Chem., vol. 26, pp. 1781-1802, 2005.

C. Plesa, “Fast translocation of proteins through solid-state nanopores,” Nano Lett., vol. 13, pp. 658-663, 2009.

D. Fologea, B. Ledden, D. S. McNabb and J. Li, “Electrical characterization of protein molecules by solid-state nanopore,” Appl. Phys. Lett., vol. 91, pp. 1-4, 2007.

A. Han, “Sensing protein molecules using nanofabricated pores,” Appl. Phys. Lett., vol. 88, pp. 28-31, 2006.

W. Li, “Single protein molecule detection by glass nanopores,” ACS Nano, vol. 7, pp. 4129-4134, 2013.

L. J. Steinbock, “Probing the size of proteins with glass nanopores,” Nanoscale, vol. 6, pp. 14380-14387, 2014.

1. A β-barrel biological nanopore having a cylindrical trans chamber, an inner constriction with a diameter of at least approximately 2 nm, and a truncated-cone cis chamber.

2. The nanopore according to claim 1, wherein the nanopore is an assembly of genetically engineered pleurotolysin (Ply) A and B subunits.

3. The nanopore according to claim 2, wherein at least one, of the residues C62 and C94 of the PlyA subunit are mutated to a non-oxydizing residue.

4. The nanopore according to claim 2 wherein the PlyB subunits comprise at least one mutation that increases the solubility of the subunit.

5. The nanopore according to claim 4, wherein the PlyB subunits furthermore comprise mutation N153D/E and/or G264K/R.

6. The nanopore according to claim 5, wherein the PlyB subunits furthermore comprise the mutation C487A/S/T.

7. The nanopore according to claim 3, wherein the PlyB subunits comprise at least one, mutation which increases the net positive charge of the inner surface of the nanopore.

8. The nanopore according to claim 7, wherein said at least one mutation is are selected from the group consisting of mutations E306K7R, E61K/R and E316K7R.

9. The nanopore according to claim 4, wherein
i) the PlyA subunits comprise the mutations C62S and C94S; and
ii) the PlyB subunits comprise the mutations N72D, A374T and A510V.

10. The nanopore according to claim 9, wherein the PlyB subunits furthermore comprise the mutations E306R, E307R and E316R.

11. The nanopore according to claim 9, wherein the PlyB subunits furthermore comprise the mutations N153D and G264R.

12. The nanopore according to claim 9, wherein the PlyB subunits furthermore comprise the mutation C487A.

13. A mutant PlyA polypeptide comprising the mutations C62A/S/T and/or C94A/S/T.

14. A mutant PlyB polypeptide, comprising the mutations N72D/E and/or A374T.

15. The mutant PlyB polypeptide according to claim 14, furthermore comprising at least one mutation selected from the group consisting of E306K7R, E61K/R and E316K7R.

16. The mutant PlyB polypeptide according to claim 14, furthermore comprising the mutations N153D/E and/or G264R/K.

17. The mutant PlyB polypeptide according to claim 14, furthermore comprising the mutation C487A/S/T.

18. A system comprising a nanopore according to claim 1, assembled into an amphipathic or hydrophobic membrane.

19. A device comprising a plurality of individual systems according to claim 18.

20. A method for providing a system according to claim 18, comprising the steps of
i) providing mutant PlyA polypeptides;
ii) providing mutant PlyB polypeptides;
iii) contacting said mutant PlyA polypeptides with liposomes or surfactant under conditions allowing for association of PlyA and liposomes or surfactant to form PlyA-liposomes; followed by
iv) contacting said PlyA-liposomes with said mutant PlyB polypeptides resulting in the formation of PlyAB lipoprotein complex; and subsequently
v) contacting the lipoprotein complex with a lipid bilayer to allow for the formation of nanopores.

21. A method of determining molecular weight, size, charge, orientation conformation, isoform or sequence of an analyte comprising translocating the analyte through the nanopore of claim 1.

22. The method according to claim 21, wherein the nanopore is subjected to an electric field such that the analyte is electrophoretically and/or electro-osmotically translocated through the nanopore, or interact with the nanopore or it is internalized inside the nanopore.

23. The method according to claim 21, wherein the analyte is a biological macromolecule or complex thereof.

24. An isolated nucleic acid molecule encoding a mutant PlyA polypeptide according to claim 13.

25. An expression vector comprising an isolated nucleic acid molecule according to claim 24.

26. A host cell comprising an expression vector according to claim 25.

27. An isolated nucleic acid molecule encoding a mutant PlyB polypeptide according to claim 14.

28. The nanopore according to claim 3 wherein one or both of C62 and C94 are mutated to A, S or T.

29. The nanopore according to claim 4 wherein the PlyB subunits comprise mutation(s) N72D/E and/or A374S/T.


SEQUENCE LISTING (8 sequences)

SEQ ID NO: 1 - DNA, 21 bp, Artificial Sequence
  primer
gattaacgccagtggtcgctc

SEQ ID NO: 2 - DNA, 19 bp, Artificial Sequence
  primer
cccacggactatcccagta

SEQ ID NO: 3 - DNA, 20 bp, Artificial Sequence
  primer
taatacgactcactataggg

SEQ ID NO: 4 - DNA, 26 bp, Artificial Sequence
  primer
cttcaggtcgaaggcttcgtcctccg

SEQ ID NO: 5 - protein, 145 aa, Artificial Sequence
  PlyA with His tag
MAYAQWVIIIIHNVGSKDVKIKNLKPSWGKLHADGDKDTEVSASKYEGTVIKPDEKLQINACGRSDAAEGTTGTFDLVDPADGDKQVRHFYWDCPWGSKTNTWTVSGSNTKWMIEYSGQNLDSGALGTITVDTLKKGSAHHHHHH

SEQ ID NO: 6 - DNA, 449 bp, Artificial Sequence
  PlyA with restriction sites
ccatggcttatgcacagtgggttattattatcattcacaatgtgggatccaaggatgtgaagattaaaaatctgaagccgtcctggggaaaattgcatgcagatggtgataaggataccgaggtatcggcgtcgaagtacgagggcactgtaattaaaccagatgaaaaattacagattaacgcctgtggtcgctcggatgcggcagagggcacgaccgggactttcgacctggttgatccggcagacggcgacaagcaggtccgccacttttactgggattgtccgtgggggtctaagacaaatacatggacggtttccggtagcaacacgaaatggatgatcgaatactccggtcagaacttagacagtggtgcgctggggaccattaccgtagatacgctgaagaaggggtcagcgcatcatcaccaccaccattgataagcttcg

SEQ ID NO: 7 - protein, 486 aa, Artificial Sequence
  PlyB with His tag
MASQAGDTLNDVIQDPTRRNKLINDNNLLKGIIMGRDGPVPSSRELIVRPDTLRAIINNRATIETTTMEAEFTETLMESNYNSASVKVSAPFITANSEYSESSSFKNTETEKSMYTSSRYLFPQGRIDFTTPDSGFDDVIKLSPQFTSGVQAALAKATGTEKREALQNLFQEYGHVFRTKVHIGGVLSAHTMETFSRSENETEVKQDVKAGLEGAVKGWGGGATAGHGNTQGTITTSQNRKLNVKYIVNGGDYTKIQNTEEWVASTNQSEHWRVIEVTEVTAVADLLPQPIRGQVKDLLKPLLGKWVDVEKVPGLESLPVSVYRPKGAIPAGWFWLGDTADASKALLVKPTLPARSGRNPALTSLHQGSGMTEQPFVDLPQYQYLSTYFGSFAHDTPPGSTLRGLRPDHVLPGRYEMHGDTISTAVYVTRPVDVPFPEDECFDLKSLVRVKLPGSGNPPKPRSALKKSMVLFDSGEKGSAHHHHHH

SEQ ID NO: 8 - DNA, 1472 bp, Artificial Sequence
  PlyB with His tag
ccatggcttcccaagcgggtgatactttgaatgacgtgatccaagatcccacccggagaaataagttaatcaatgacaataacttacttaaggggatcataatggggagagacgggccagtcccttcatcgagagaattgatagtccgtccggacactctgcgggcaataatcaacaaccgcgccacgatagagactaccactatggaggccgaatttacggaaacgcttatggagtcgaattacaattcagcgtcggtgaaagttagcgcacctttcataacagcaaactcagaatatagcgaatcctcgtccttcaagaacacggagactgaaaagtccatgtacaccagttctagatatttgtttccgcaaggtcgcattgattttacaaccccagactcggggttcgatgacgttattaagctatcacctcaattcacaagcggagttcaagcagcattggcaaaggccacagggaccgaaaaacgggaagcgcttcagaacctgttccaagagtatggacacgtgttcagaactaaagttcatatcgggggcgttttaagtgctcatacaatggagacgttcagcagaagcgagaatgagacagaagtcaaacaagacgtcaaagctgggcttgagggcgctgttaaggggtggggaggtggcgcaaccgcgggacatggcaacactcaagggaccattactactagccaaaaccgcaaattgaatgtaaaatacatcgtgaatggaggcgattacacaaaaattcagaataccgaggagtgggtagcgtctacgaaccagtccgaacattggcgtgtaatcgaagttaccgaggtaaccgctgttgcagatttactgccacagcctattcgtgggcaagtcaaggatcttcttaaaccgttgcttgggaagtgggtagatgtagagaaagtgcctggtttagagtcacttcctgtatccgtatatcgcccgaaaggggccattcccgccggatggttttggttaggcgatactgctgacgcttccaaagcccttcttgtcaaaccaacattaccggctcggtctggacgtaaccctgcgcttacgagccttcaccaggggtcaggtatgacggaacaaccgttcgttgatttgcctcagtatcaatacctttcgacatatttcggaagttttgcgcatgataccccgccaggatcgactcttcgtggtttgcgcccggatcacgtcctgccaggccggtacgaaatgcatggagacaccatatctacggcggtctatgtaacacgtcccgtggatgtaccattcccggaggacgaatgcttcgacctgaagagcttggttcgtgttaagttgcccgggtccgggaatcctccaaaaccccgtagcgcgttgaagaaatcaatggtgctgttcgattcaggtgaaaaagggtcagcgcatcatcaccaccaccattgataagcttcg